(19) United States
(12) Patent Application Publication
(10) Pub. No.: US 2005/0265956 A1
(43) Pub. Date: Dec. 1, 2005

(54) Polyalkyleneimine-graft-biodegradable polymers for delivery of bioactive agents

(76) Inventors: Ye Liu, Singapore (SG); Kok Hou Wong, Singapore (SG); Guobin Sun, Singapore (SG); Chaobin He, Singapore (SG); Kam W. Leong, Ellicott City, MD (US)

Correspondence Address: KLARQUIST SPARKMAN, LLP, 121 SW SALMON STREET, SUITE 1600, PORTLAND, OR, 97204, US

(21) Appl. No.: 11/036,902

(22) Filed: Jan. 12, 2005

Related U.S. Application Data
(60) Provisional application No. 60/535,506, filed on Jan. 12, 2004.

(51) Int. Cl. A61K 38/17; C08G 63/91

(52) U.S. Cl. 424/78.27; 525/54.1

(57) ABSTRACT

The invention provides poly(alkyleneimine)-graft-biodegradable polymers and methods for preparing such polymers. The poly(alkyleneimine)-graft-chitosan polymers may optionally contain a targeting element. The poly(alkyleneimine)-graft polymers may be used to deliver a bioactive agent into a cell.

### CROSS-REFERENCE TO RELATED APPLICATION

This application claims priority from U.S. provisional application No. 60/535,506 filed Jan. 12, 2004, which is fully incorporated by reference.

### FIELD OF THE INVENTION

The invention relates generally to poly(alkyleneimine) graft copolymers and to the use of such polymers in delivering bioactive agents into target cells in vivo and in vitro.

### BACKGROUND OF THE INVENTION

Gene therapy focuses on the delivery of exogenous genes to cells in need of such therapy. Initial attempts at developing nucleic acid vectors exploited viral gene-delivery methods capable of delivering exogenous DNA into cells with both great efficiency and specificity. Generally, these methods employ recombinant non-replicative viral vectors. However, the use of viral-based delivery vectors have a number of drawbacks, including, the high immunogenicity of the viral coat. In certain instances, the production of viral-based delivery systems requires the provision of a replication competent “helper virus”, and preparing compositions of the gene delivery vector that are free of helper virus may be problematic. Furthermore, viral-based gene delivery systems also have the risk that the delivery vector may become replication competent and perhaps even pathogenic or tumorogenic, for instance, through recombination with a replication-competent helper virus.

Synthetic gene delivery systems have been developed with the aim of minimizing or avoiding the risk posed by viral gene delivery systems. In order to be an effective gene delivery system, the vector must condense DNA, protect the DNA from nucleases, favor its cellular uptake and allow the release of the targeted DNA into the cell nucleus (Kichler (2004), J Gene Med 6:S3-S10).

To date, two general classes of synthetic gene delivery systems have been developed: cationic lipids and cationic polymers. Cationic liposomes generally have the disadvantages that DNA may be degraded in the liposomes and cationic lipids may have strong cytotoxicity (Kim et al (2001) Bull. Korean Chem. Soc. 22(10): 1069).

Cationic polymers function by forming self-associating “polyplexes” with DNA. The polyplexes are believed to be stabilized by electrostatic interactions between the negatively charged phosphates of the nucleic acid backbone and the positively charged groups on the cationic polymer. Poly(lysine) was the first cationic polymer used to mediate cell transfection (Wu & Wu (1987), J. Biol. Chem. 262:4429). More recently, poly(alkyleneimine)s, (PAI) in particular poly(ethyleneimine) (PEI), have proven to be versatile and effective synthetic delivery vectors both in vitro and in vivo. (Boussif et al. (1995), Proc. Nat. Acad. Sci. 92:7297). PEI is commercially available in linear and branched forms in a wide variety of molecular weights. Branched PEI contains primary, secondary and tertiary amine groups, which may be present in about a 1:1:1 or a 1:2:1 ratio (Kunath et al. (2003), Controlled Release 89:113).

Although the exact details of the cellular uptake of cationic polymer-DNA polyplexes is not fully known, polyplexes are believed to enter the cell through endocytosis. In the case of PEI/DNA polyplexes, the large number of protonatable nitrogens in the PEI backbone is believed to increase transfection efficiency by disrupting the integrity of the endosomes by acting as a so-called “proton sponge”, thereby facilitating endosomal escape of the polyplex (Godbey et al J. Control Release (1999) 60:149).

Chitosan has also emerged as a possible cationic polymer for the delivery of nucleic acids. High molecular weight chitosans may form stable complexes with DNA. The complexes generally display a wide range of sizes dominated by aggregates. High molecular weight chitosans are often sparingly soluble at physiological pH and are viscous at concentrations necessary for gene delivery. Lower molecular weight chitosans (1.2 to 4.7 kDa) can function as gene delivery vectors. The efficiency of gene delivery is dependent on the charge density of the chitosan polymer. In order to be an effective delivery vehicle, the number of monomer units should be at least 6 and the degree of acylation should be less than about 35% (Köping-Höggård et al. (2003), J. Gene Med. 5: 130). Oligo-chitosan with 24 monomer units gave a level of gene expression similar to that of high molecular weight chitosan, in vitro and in vivo (Köping-Höggård et al. (2003), Gene Med. 5: 130).

The transfection efficiency of PEI-polyplexes has been shown to depend on the molecular weight of the polycation. PEI having an average molecular weight of about 25 kDa has both high transfection efficiency and cytotoxicity. PEI with an average molecular weight of less than about 1.8 kDa has low cytotoxicity and little to no transfection efficiency (Godbey et al. (1999), J. Biomed. Mater. Res. 45:268; Fischer et al. (1999) Pharm Res. 16: 1273; Ahn et al (2002), J. Control. Release 80:273; Kunath et al. (2003) J. Control Release 89:113). Further, as PEI is not biodegradable, higher molecular weight PEIs may not be safe for the long-term treatment of a patient.

One approach to reduce the cytotoxicity of PEI, improve its biocompatibility but still keep its high transfection efficiency has been to combine low molecular weight oligo-PEIs with some kind of linker to form higher molecular weight oligo-PEI copolymers. For example, linking PEI to non-ionic water soluble polyethers, such as poly(ethyleneoxide) (PEO) and polyethylene glycol (PEG), have been shown to reduce the toxicity of the cationic polymer (Nguyen et al. (2000), Gene Therapy 7:126; Choi et al. (2001) Bull Korean Chem Soc. 22(1):46; Kichler (2004), J. Gene Med. 6:S3). The higher molecular weight oligo-PEI copolymers are expected to possess high transfection efficiency and to degrade into PEIs of lower molecular weights which is expected to reduce the cytotoxicity of the oligo-PEI polymers. A problem with this approach is that the resulting oligo-PEI copolymers are often sparingly soluble and may result in a gel (Ahn et al. (2002), J. Control Release 80:273). Even if soluble products are obtained, for example by carefully adjusting the reaction conditions, it is often difficult to control the structures of the products such as the molecular weights and constituents and the reproducibility is poor.

Further, while this approach has been shown to be effective in reducing cytotoxicity, some of the linkers that have been employed are themselves not biodegradable. Cumulative cellular exposure time plays a primary role in the cytotoxicity of slowly degradable or non-degradable polycations (Putnam & Langer (1999), Macromolecules 32:3658). One of the most important demands on macromolecular drug carriers is that they must not accumulate in the body (Petersen et al. (2002), Bioconjugate Chem 13:812). As a result, non-biodegradable cationic polymers may not be suitable when repeated administration is required over a relatively short period of time.

Ahn et al. ((2002), J. Control Release 80:273) disclose cross-linked biodegradable PEG-PEI copolymers produced by treating PEI with a bifunctional PEG succinimidyl succinate (SS-PEG). Due to the chemical structures of PEI, the reaction between PEI and SS-PEG generally resulted in non-soluble copolymers. The authors disclose that it was possible, in some instances, to produce water-soluble PEI-co-PEG polymers only after decreasing the concentration of the reactants. In other instances, decreasing the initial concentration of SS-PEG and PEI was not enough to obtain a water-soluble PEI-co-PEG copolymer, probably due to the increased probability of cross-linking.

Choi et al. ((2001), Bull. Korean Chem. Soc.) 22(1):46) disclose PEG-graft-PEI copolymers synthesized by conjugating 25 kDa PEI (PEI 25K) with monofunctional and bifunctional low molecular weight (550 and 600 Da) carboxylated-PEG derivatives. In each case, PEG is grafted to the PEI main chain through an amide bond. The PEG-graft-PEI copolymers displayed reduced cytotoxicity relative to PEI alone and the biocompatibility of the PEG-graft-PEI copolymers increased with increasing degrees of PEGylation. The PEG-graft-PEI polymers increased the water solubility of polyplexes containing plasmid DNA. Polyplexes comprising the monofunctional PEG-graft-PEI copolymer had reduced transfection efficiencies in two cells lines, whereas PEG-graft-PEI copolymers formed from the bifunctional PEG derivatives had transfection efficiencies comparable to that of PEI 25K. The disclosed polymers have some limits in the extent of grafting and in the degree of modification.

U.S. Pat. No. 6,586,524 discloses a cationic polymer wherein 1% to 10% of the cationic groups of PEI are grafted with PEG which is, in turn, covalently bound to a targeting moiety, specifically galactose. Relative to PEI, the galactose-PEG-PEI polymers substantially increased the solubility of DNA polyplexes. Gal-PEG-PEI/DNA polyplexes wherein 1 mole percent of the PEI amine groups are grafted with GAL-PEG have a greater transfection efficiency than PEI 25K.

U.S. Pat. No. 6,696,038 discloses a biodegradable cationic lipopolymer having reduced in vitro and in vivo toxicity comprising branched PEI, a cholesterol derived lipid anchor and a biodegradable linker which covalently links the PEI and the lipid anchor.

U.S. 2001/0005717 discloses PEI polymers that have been modified by the covalent attachment of hydrophilic polymers, including PEG, polyvinylpyrrollidones, polyacrylamides, polyvinylalcohols or copolymers of these polymers. The polymers disclosed in the application are optionally modified by coupling a ligand to the PEI/hydrophilic polymer.

Gosselin et al. ((2001), Bioconjugate Chem.12:989) disclose 800-Da PEI cross-linked with the homobifunctional amine-reactive reducible cross-linking reagents, dithiobis(succinimidylpropionate) and dimethyl-3,3′-dithiobispropionimidate. The cytotoxicity of the obtained polymers was significantly reduced but their transfection efficiencies were several times lower than PEI 25K.

Petersen et al. ((2002), Bioconjugate Chem. 13(4):812) disclose a 1200 Da PEI-co-(oligo L-lactic-co-succininc acid)) polymer (“P(EI-co-LSA”). The molecular weight of the resulting polymer suggested that the oligo-L-lactic-co-succinic acid reacted with PEI only to modify and graft the polycation but did not link different PEI macromolecules together. P(EI-co-LSA), similar to low molecular weight PEI, displayed low cytotoxicity and increased the transfection efficiency of 1200 Da PEI at an N/P ratio of 50. The transfection efficiency of P(EI-co-LSA) was not compared to PEI 25K.

Forrest et al. ((2003), Bioconjugate Chem. 14(5):934) combined 800-Da PEI with diacrylate linkers to produce highly cross-linked polymers comprising several hundred PEI monomers. Relative to PEI 25K, the resulting cross-linked PEI polymers showed lower cytotoxicity and had 2 to 16-fold higher transfection efficiencies.

### SUMMARY OF THE INVENTION

The invention provides PAI-graft-biodegradable polymers which can be used to deliver bioactive agents and may be particularly useful in gene therapy.

In one aspect, the invention provides a poly(alkyleneimine)-graft-biodegradable polymer comprising at least two poly(C₂-C₆ alkyleneimine) side chains and a biodegradable polymer main chain, wherein each of the at least two side chains is linked to the main chain by a single covalent bond.

In another aspect, the invention provides a poly(alkyleneimine)-graft-biodegradable polymer comprising the structure of formula I: 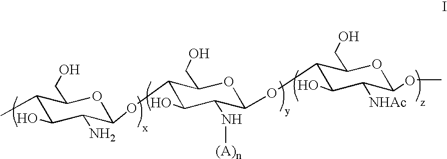
wherein:
x+y+z is 2 to about 1000;
x/(x+y+z) is 0 to about 99.9%;
y is at least 2;
z/(x+y+z) is 0 to about 60%; and
(A)ₙ is a poly(C₂-C₆ alkyleneimine) having n repeating units.

In yet another aspect, the invention provides a method of preparing a PAI-graft-biodegradable polymer comprising the step of reacting a C₂-C₆ alkyleneimine and a biodegradable polymer under acidic conditions.

In yet another aspect the invention provides a composition comprising a bioactive agent and a PAI-graft-biodegradable polymer according to various embodiments of the invention.

Other aspects and features of the present invention will become apparent to those of ordinary skill in the art upon review of the following description of specific embodiments of the invention in conjunction with the accompanying figures.

### BRIEF DESCRIPTION OF THE DRAWINGS

In the figures, which illustrate by way of example only, embodiments of the present invention:

FIG. 1 depicts possible reaction schemes for the formation of PEI-graft-chitosan.

FIG. 2 depicts ¹H-NMR spectra of oligo-chitosan (FIG. 2a), PEI-graft-chitosan (FIG. 2b) and hexadecane-graft-PEI-graft-chitosan (FIG. 2c).

FIG. 3 depicts ¹³C-NMR spectra of oligo-chitosan (FIG. 3a) and PEI-graft-chitosan (FIG. 3b).

FIG. 4 depicts gel permeation chromatographs of oligo-chitosan (FIG. 4a), PEI-graft-chitosan (FIG. 4b) and hexadecane-graft-PEI-graft-chitosan (FIG. 4c).

FIG. 5 depicts the results of a gel-retardation assay of pCMV-Luc plasmid DNA by PEI-graft-chitosan (FIG. 5A) and hexadecane-graft-PEI-graft-chitosan (FIG. 5B) at various N/P ratios.

FIG. 6 is a comparison of the cytotoxicity profiles of PEI-graft-chitosan (a), hexadecane-graft-PEI-graft-chitosan (b), poly(lysine) (c) and PEI 25K (d).

FIG. 7 is a comparison of the transfection efficiencies of PEI-graft-chitosan/DNA polyplexes, hexadecane-graft-PEI-graft-chitosan/DNA polyplexes, PEI polyplexes and naked DNA in HeLa cells.

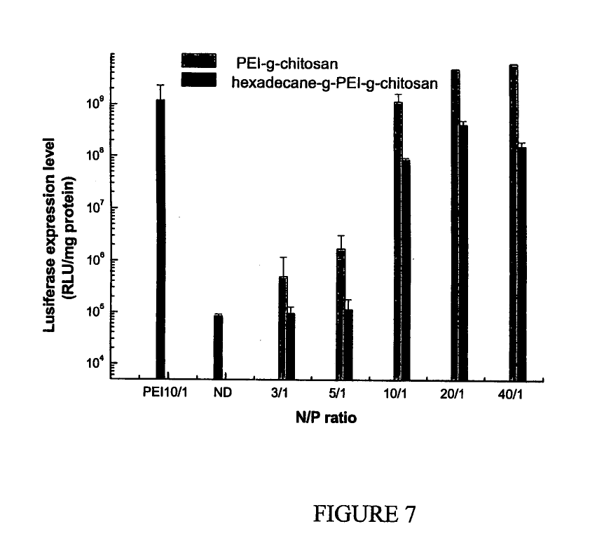

FIG. 8 depicts luciferase gene expression in a number of organs 3 days after administration of PEI-graft-chitosan/DNA polyplexes of varying N/P ratios.

FIG. 9 depicts luciferase gene expression in different lobes of a transfected rat liver 3 days after administration of PEI-graft-chitosan/DNA polyplexes.

FIG. 10 compares the relative luciferase transgene expression of a several PEI-graft-chitosan/DNA polyplexes, naked DNA, a PEI/DNA polyplex and a chitosan/DNA polyplex in the liver.

### DETAILED DESCRIPTION

There is provided a poly(alkyleneimine)-graft-biodegradable polymer and in different embodiments comprises at least two poly(C₂-C₆ alkyleneimine) side chains linked to a biodegradable polymer main chain wherein each side chain of the graft polymer is linked to the main chain through a single covalent bond.

As used herein, “graft polymer” refers to a polymer with one or more species of blocks connected to the main chain as side chains, the side chains having constitutional or configurational features that differ from those of the main chain (Glossary of Basic Terms In Polymer Science IUPAC recommendations 1996). As used herein, “blocks” refers to a portion of a macromolecule comprising many constitutional units that has at least one feature which is not present in the adjacent portions (Glossary of Basic Terms In Polymer Science IUPAC recommendations 1996). As used herein, “main chain” refers to the linear chain to which all other chains, long or short, may be regarded as being pendant, and “side chain” refers to an oligomeric or polymeric offshoot from a macromolecular chain (Glossary of Basic Terms In Polymer Science IUPAC recommendations 1996).

The biodegradable polymer main chain may be a natural or synthetic biodegradable polymer. In certain embodiments, the biodegradable polymer may be a polysaccharide, a polyester, a polyamide, a poly(ester amide), a poly(ester carbonate), a poly(ester ether), a poly(ester urethane), a polypeptide, a polyurethane, a polyphosphoester, a poly(phosphazenes) homopolymer, or a copolymer comprising one or more of these monomer units. In a specific embodiment, the biodegradable polymer is poly(L-aspartic acid-co-PEG). In various embodiments, the number of the repeating units of the biodegradable polymer main chain is about 2 to about 10,000.

In some embodiments, the PAI side chain is a PEI side chain. The molecular weight of the PEI side chain may be about 100 Da to about 100 kDa, which roughly corresponds to PEI side chains with about 2 to about 2325 repeating ethyleneimine units. The PEI side chains may be linear or branched.

In various embodiments, the PAI-graft-biodegradable polymer may further comprise a targeting element. As used herein “targeting element” refers to any element that may facilitate or mediate or enhance the delivery of an associated molecule or macromolecular complex to a particular cell, collection of cells, nuclei, tissue or promote the endocytosis, phagocytosois or endosomal escape of the associated molecule. The targeting element may be a ligand (or a fragment thereof) of a normally expressed cell-surface receptor. In certain embodiments, the targeting element is a protein, glycoprotein, lipoprotein or an antibody or an antibody fragment directed against a cell-surface epitope of the target cell. The antibody or antibody fragment may be, or be derived from, a polyclonal antibody, or more preferably from a monoclonal antibody. Examples of targeting elements would be well known to a person skilled in the art, (for example, reviewed in Molas et al (2003), Current Gene Therapy 3:468) and include, among other things, transfernin (Curiel et al. (1991), Proc. Nat. Acad. Sci 88: 8850; asialoorosomucoid (ASOR) (Christiano et al. (1993), Proc. Nat. Acad. Sci 90(6):2121; Wilson et al. (1992) J. Biol. Chem. 267(2):963), mannose (Dieboldt et al. (1999), J. Biol. Chem. 274(27): 19087), galactose, an anti-CD3 antibody (Ogris et al. (2001), AAPS PharmSci 3(3) article 21), an anti-HER-2 antibody (Foster & Kern (1997), Hum Gene Ther. 8(6):719) cholesterol or myristate (Kim et al. (2001), Bull Korean Chem Soc. 22(10):1069). In specific embodiments, the targeting element is a transferrin, an asiaglycoprotein, a HIV gp120 envelope protein or sialic acid. In other embodiments, the targeting element is cholesterol or a C₅-C₂₀ alkyl group. As used herein, an “alkyl group” refers to a linear or branched, straight or cyclic or polycyclic alkyl group derived from a linear or branched, straight or cyclic alkane by the removal of a hydrogen atom. In another specific embodiment, the targeting element is a C₁₆ alkyl group.

A person skilled in the art would understand, based on the particular application, which targeting element would be most appropriate. For example, if hepatic cell targeting is desired, targeting elements that can specifically bind to receptors present on hepatic cells, such as, for example, α-2-macroglobulin (Schneider et al (1996) Nucl. Acid Res. 24:3873) may be advantageously used. Targeting element for other types of cells would be known to a person skilled in the art (see, for example, Molas et al. (2003), Current Gene Therapy 3:468). An appropriate targeting element may be chosen by preliminary tests that compare the in vitro transfection efficiency of polyplexes with a specific targeting elements to those of polyplexes containing a different or no targeting element.

In various embodiments, the poly(alkyleneimine)-graft-biodegradable polymer is a poly(alkyleneimine)-graft-chitosan polymer comprising the structure of formula I: 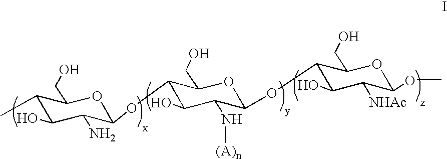
wherein:
x+y+z is 2 to about 1000;
x/(x+y+z) is 0 to about 99.9%;
y is at least 2;
z/(x+y+z) is 0 to about 60%; and
(A)ₙ is a poly(C₂-C₆ alkyleneimine) having n repeating units.

With reference to formula I, the “main chain” refers to the horizontally extending poly(D-glucosamine) backbone and the “side chains” are the pendant (A)ₙ groups. Formula I is not intended to depict a periodic polymer wherein the x, y, and z blocks are arranged in any specific order. Rather, the poly(alkyleneimine)-graft-chitosan polymers of formula I encompass random PAI-graft-poly(D-glucosamine) polymers, where the main chain blocks represented by x, y and z may be joined in any order, and includes PAI-graft-polymers wherein x is 0, z is 0 or (x+z) is 0. Similarly, formula I is not intended to depict a polymer where each (A)ₙ side chain has an identical number of repeating units. Rather, the n in (A)ₙ represents the average number of PAI repeating units for the poly(alkyleneimine)-graft polymer sample. The value of n in a given polymer comprising formula I may be determined by known methods, for example, NMR spectroscopy.

The poly(C₂-C₆ alkyleneimine) side chain represented by (A)ₙ may be linear or more preferably branched. In certain embodiments, (A)ₙ is a branched polymer having primary, secondary an tertiary amine groups. In various embodiments, the PAI is PEI. In specific embodiments, the (A)ₙ is branched PEI.

In various embodiments, n is about 2 to about 2325. In specific embodiments, (A)ₙ is PEI wherein n is about 2 to about 2325.

As would be understood by a person skilled in the art, the greater the value of n the more strongly (A)ₙ will interact with a polyanion, such as, for example, DNA, such that stable polymer complexes may be formed using lower relative amounts of the polycationic polymer. Therefore, n is not particularly limited and suitable values of n required to form a stable complex with a polyanion may be determined by those skilled in the art.

As would be further appreciated by a person skilled in the art, the PAI-graft-polymer of formula I can also have differing degrees of deacetylation. Within the context of formula I, the degree of deacetylation is given by (x+y)/(x+y+z). Generally, increased levels of deacetylation increase the solubility of the chitosan polymer in dilute acidic media. Chitosans, which result from the deacetylation of chitin, are commercially available with varying degrees of acetylation or may be prepared by methods known in the art. The degree of deacetylation may be determined by methods known to person skilled in the art, for example, such as by hydrogen bromide titrimmetry (Sabnis & Block (1997) Polym Bull 39:67), infrared spectroscopy (Sabnis & Block (1997) Polym Bull 39:67), first-derivative UV-spectrophotometric analysis (Tan et al (1998) Talanta 45:713) or a ninhydrin assay (Sarin et al (1981) Anal Biochem 117:147). In certain embodiments, the degree of deacetylation is about 10% to about 100%, preferably about 40% to about 98%, more preferably about 50% to about 95% and most preferably about 75% to about 95% and the term “chitosan”, as used herein, is intended to refer to chitin/chitosan with such varying degrees of deacetylation.

In certain embodiments, the poly(alkyleneimine)-graft polymer of formula I further comprises a targeting element. The targeting element may be linked directly to an atom of the PAI-graft polymer of formula I or may be linked to the PAI-graft polymer by a linker or spacer. The targeting element may be attached to a free amino group of the main chain, or more preferably to a nitrogen of the side chain poly(alkyleneimine). Generally, the targeting element is covalently linked to the PAI-graft-polymer.

Synthesis of poly(alkyleneimine)-graft-biodegradable polymers

PAI-graft-biodegradable polymers may be prepared by reacting alkyleneimine monomers with a biodegradable polymer, such as, for example, poly(L-aspartic acid-co-PEG) under acidic conditions. PAI contains multiple amines, and the reaction between pre-formed PAI and a multifunctional biodegradable polymer may result in a non-soluble cross-linked network copolymer (Ahn et al. (2001) J. Controlled Release 80:273). Without being limited to any particular theory, it is believed that polymerizing alkyleneimine monomers in the presence of a biodegradable polymer may prevent the formation of network or cross-linked PAI-biodegradable polymers and provide a soluble PAI-graft polymer. As used herein, “acidic conditions” refers to a solution with a pH of less than 7, preferably less than about 6, more preferably less than about 5.

In specific embodiments, polymers according to formula I may be synthesized by an acid-catalyzed cationic polymerization of alkyleneimines in aqueous solution in the presence of chitosan. Such polymerization may be effected by reacting an acidified chitosan solution and an alkyleneimine solution. The chitosan solution may be acidified by a concentrated mineral acid, such as, for example, concentrated (35% w/v) HCl. In various embodiments, HCl is added to the chitosan solution in such amounts that the molar ratio of hydrochloric acid to the amine in chitosan is about 1:60 to about 20:1, preferably 1:40 to about 1:5, and more preferably about 1:25 to about 1:5.

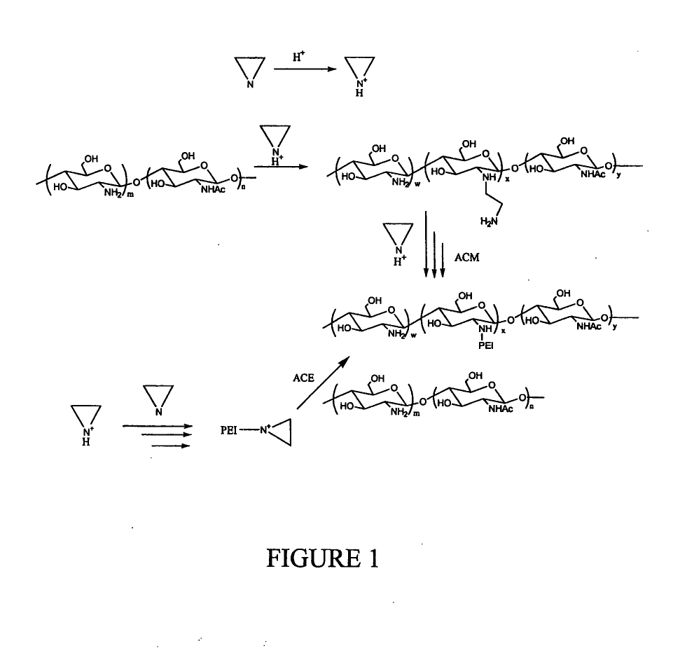

FIG. 1 illustrates two possible mechanisms for forming PEI-graft-chitosan, the active monomer (AM) and active chain end (ACE) mechanisms. In the AM mechanism, the activated monomer is added to the free amines in chitosan or PEI chains grafted to chitosan. In the ACE mechanism, a PEI polymer chain is formed in solution and this chain is transferred to the free amines of chitosan. Hybrid mechanisms comprising portions of the ACE and AM mechanisms are also contemplated. For example, ethyleneimine monomers may be polymerized by the AM mechanism onto PEI chains covalently attached to the chitosan backbone via the ACE mechanism.

Chitosan solutions are generally polydisperse having a distribution of chain lengths and therefore molecular weights. Chitosan of varying chain lengths or molecular weights may be obtained from a number of commercial sources (Sigma, Aldrich or BASF) or may be prepared by the deacetylation of chitin, which is also commercially available, such as, for example, by exposing chitin to concentrated alkali at high temperature. Low molecular weight chitosan is commercially available or may be prepared from high molecular weight chitosan via degradation. In this context, the low molecular weight chitosan may be prepared by methods known in the art, such as, for example, by enzymatic hydrolysis (Köping-Höggård et al. (2003), J Gene Med. 5:130), acid hydrolysis (Vårum et al. (2001), Carbohydrate Polymers 46:89), or nitrous acid degradation (Allan et al. (1995), Carbohydrate Res. 227:257; Tommeraas et al. (2001), Carbohydrate Res 333:137). Preferably, low molecular weight chitosan is prepared by degrading high molecular weight chitosan by H₂O₂ (JP O₂-22301). In specific embodiments, the chitosan used to prepare the PAI-graft-polymer of formula I has a viscosity (Brookfield, 1% acetic acid) of 20 to 200 cps.

To prepare a chitosan solution, chitosan may be dissolved in aqueous media, at a concentration about 1% to about 50% w/v preferably about 3% to about 20%, more preferably about 5% to about 10%. As would be known to a person skilled in the art, higher molecular weight chitosan solutions may require the addition of an acid in order to be soluble in an aqueous solvent.

In certain embodiments, chitosan has a molecular weight (in Da) of about 200 to about 1,000,000, preferably about 200 to about 10,000, more preferably about 500 to about 9,000, and most preferably about 1500 to about 6000.

Ethyleneimine may be prepared from ethanolamine by the method of Wenker ((1935), J. Am. Chem. Soc. 57: 2328) or by other known methods (U.S. Pat. No. 4,568,747). The boiling point of the prepared ethyleneimine is preferably between about 55.0 and about 56.0° C. Other alkyleneimines, such as, for example, propyleneimine (2-methylaziridine) or butyleneimine (2-ethylaziridine) may be prepared by analogous processes.

In some embodiments, the C₂-C₆ alkyleneimine solution is added dropwise to the acidified chitosan solution in such amounts that the molar ratio of ethyleneimine to chitosan amine is about 0.1 to about 500, preferably about 1 to about 100, more preferably about 2 to about 20, and most preferably about 3 to about 10. Preferably, the ethyleneimine solution is added dropwise with stirring to the acidified chitosan solution. Without being limited to any particular theory, it is believed that the high molar ratio of amino groups in chitosan to hydrochloric acid reduces ethyleneimine homopolymerization.

The grafting/polymerization reaction may be performed between about −20° C. and about 100° C. More preferably, the reaction is performed between about −10C and about 90° C., more preferably between about 0° C. and about 80° C., and even more preferably, between about 20° C. and about 70° C. The reaction may be incubated for any period of time sufficient to obtain the PAI-graft-polymer of formula I, for example, about 10 hours to about 10 days.

The grafting/polymerization reaction may be monitored by methods known to a person skilled in the art, for example, by monitoring aliquots of the reaction by ¹³C or ¹H NMR spectroscopy (Ahn et al. (2001), J. Controlled Release 80:273). For example, the molar ratios of the poly(alkyleneimine) to chitosan in the PAI-graft-chitosan polymer may be determined by comparing the relative peak areas of NMR signals from ¹H or ¹³C nuclei in the poly(alkyleneimine) side chains and the chitosan backbone. The ratio of primary, secondary and tertiary amines in PAI may be determined by methods known in the art, for example, by ¹³C NMR analysis (von Harpe et al. (2000) J. Control Release 69:309).

The structures of the PAI-graft-chitosan polymers obtained according to above method can be controlled. For example, the length of the grafted PAI chains and the ratio of PAI to chitosan in the resulting PAI-graft-chitosan polymer may be adjusted by controlling the feed amount of the alkyleneimine.

The PEI-graft-chitosan polymers prepared by these methods may be soluble in aqueous solutions and may optionally be further purified by methods known in the art, including, but not limited to, one or more of dialysis, precipitation, crystallization, chromatography, drying under vacuum, filtration and the like.

Without being limited to any particular theory, the primary, secondary and tertiary amines contained in the PAI-graft-polymer may provide sufficient positive charges for the adequate condensation of DNA. The biodegradable chitosan backbone and the relatively low molecular weight of PAI in the PAI-graft-chitosan polymers may reduce the cytotoxicity of the polymers, a property that is desirable for polymers intended for use as a vector for delivering bioactive agents to cells and tissues in vivo and in vitro.

A targeting element may be covalently attached to one or more of the free amino group of chitosan or PEI-graft-chitosan by methods known in the art. For example, chitosan may be partially substituted by lactose residues by reductive amination in the presence of lactose and sodium cyanoborohydride (Erbacher et al. (1998), Pharmaceutical Research 15(9):1332) and the degree of lactose substitution determined by the resorcinol sulfuric acid micromethod (Monsigny et al. (1988), Anal. Biochem 175:525).

In some embodiments, one or more targeting elements may be attached to one or more poly(alkyleneimine) amino groups. For example, the targeting element may be covalently linked by a bifunctional linker such as 1-ethyl-3-(3-dimethylaminopropyl) carbodiimide (Christaino et al. (1993), Proc. Nat. Acad. Sci 90:2122) or an appropriately modified monofunctional ligand, such as, for example, cholesterol chloroformate (Kim et al. (2001), Bull Korean Chem Soc. 22(10):1069). Polypeptide-based targeting elements may be covalently linked to the poly(alkyleneimine) as described, for example, in WO 93/07283.

PAI-graft-Biodegradable Polymer Polyplexes

The PAI-graft-biodegradable polymers according to various embodiments of the invention may be used, for example, to deliver bioactive agents by forming electrostatic complexes, or “polyplexes” with anionic or neutral bioactive agents. In various embodiments, the PAI-graft-biodegradable polymer comprises the structure of formula I.

As used herein, bioactive agents include therapeutic, diagnostic or prophylactic agents. The bioactive agent may be, for example, a small molecule, organometallic compoTunds, polynucleotide, polypeptide, polynucleotide metal, an isotopically labeled chemical compound, drug, vaccine, immunological agent, and the like. Prophylactic agents include, but are not limited to, antibiotics, nutritional supplements, and vaccines. Vaccines may comprise isolated proteins or peptides, inactivated organisms and viruses, dead organisms and viruses, genetically altered organisms or viruses, and cell extracts. Diagnostic agents include, but are not limited to, gases, metals, commercially available imaging agents used in positron emission tomography (PET), computer assisted tomography (CAT), x-ray, fluoroscopy, and magnetic resonance imaging (MRI), as well as contrast agents. Examples of suitable materials for use as contrast agents in MRI include gadolinium chelates, as well as iron, magnesium, manganese, copper, and chromium or their chelates. Examples of materials useful for CAT and x-ray imaging include iodine-based materials. The agent may be described as a single entity or compound or a combination of entities or compounds.

While the bioactive agent exemplified in the Examples is DNA, as will be appreciated by a person skilled in the art, cationic polymers may be used to deliver other neutral or negatively charged molecules into a cell.

In various embodiments, the bioactive agent may be a polynucleotide. As used herein, “polynucleotide” includes, but is not limited to DNA, RNA, DNA/RNA hybrids, and derivatives of DNA or RNA, including modification in the bases, sugars, and/or the phosphate linkage. Unless the context dictates otherwise, “polynucleotide” is herein used interchangeably with “nucleic acid”. Polynucleotides may be linear or more preferably circular. The polynucleotides may be single stranded, triple stranded or more preferably double stranded. In various embodiments, the polynucleotide may be about 500 to about 10000 bases (or base pairs in the case of a double stranded polynucleotide), and more preferably about 1000 to about 5000 bases (or base pairs in the case of a double stranded polynucleotide). The polynucleotide may be chemically synthesized by known methods, such as, for example, solid-phase phosphoramidite synthesis or be obtained from a variety of commercial sources. More preferably the polynucleotide is a recombinant polynucleotide that may be propagated and isolated from an appropriate host, such as for example, bacteria or yeast, by methods known in the art, for example, those described in Sambrook et al. Molecular Cloning, A Laboratory Manual (3^{rd} ed) Cold Spring Harbour Laboratory Press (2001) and other laboratory manuals.

In other embodiments, the biaoctive agent is RNA. The bioactive RNA maybe a small interfering RNA (siRNA), which when introduced into a cell may mediate targeted suppression of gene expression. The RNA contained within a polyplex may possess catalytic activity, for example, a ribozyme. In some embodiments, the RNA may be a mRNA that may be directly translated to produce a polypeptide. In other embodiments, the RNA may be a sense RNA having a sequence that is substantially identical to an endogenous target gene or mRNA sequence, or antisense RNA that is substantially complementary to an endogenous gene sequence or mRNA sequence. The RNA may be prepared by known methods, for example, chemical synthesis, or through known molecular biology techniques such as, for example, in vitro translation or may be purified from a natural or heterologous source by methods known in the art, such as, for example those accompanying commercial RNA purification kits such as, for example, RNEasy (Qiagen).

In preferred embodiments, the polynucleotide is DNA. Preferably, the DNA is a double stranded circular plasmid and more preferably a double stranded circular expression vector. As used herein, “expression vector” refers to a polynucleotide capable of directing the transcription and translation of a target genetic coding region in one or more hosts. Preferably, the target coding sequence is engineered to take advantage of the codon bias of the specific host.

In addition to the target genetic coding region, expression vectors generally contain additional elements that direct or enhance protein expression, such as, for example, promoters, enhancers, ribosome binding sites, TATA boxes, transcriptional termination sequences and the like. The promoters may be constitutive or inducible. The promoters may be ubiquitous in that they are capable of driving the expression of the target gene in a number of cell types within a host, such as, for example, a CMV promoter, or may drive expression in restricted cell-types, for example, hepatic cells, such as for example, ApoCIII promoter. Examples of other ubiquitous and cell-specific promoters would be known to a person skilled in the art.

In other embodiments, the polynucleotide is designed to effect targeted gene replacement. In these embodiments, the polynucleotide is preferably linear DNA. For example, the polynucleotide may comprise a wild-type gene sequence that may optionally contain promoter and enhancer regulatory elements. Without being limited to any particular theory, the polynucleotide, once introduced into the target cell, may integrate into the host cell's genome, for instance, through homologous recombination. Polynucleotides according to this embodiment may be advantageously used to replace copies of defective or mutant endogenous genes with functional or preferably fully functional exogenous gene sequences.

In other embodiments, the bioactive agent may be a polypeptide. As used herein, “polypeptide” refers to a polymer of amino refers joined by peptide bonds between the alpha carboxyl group of one residue of the backbone alpha amino group of the next reside. Polypeptides may be circular, or more generally linear, and may optionally contain intermolecular or intramolecular covalent cross-links, for example between two cysteine residues.

As used herein, “amino acid” refers to any one of the nineteen genetically encoded L-amino acids (alanine, threonine, serine, cysteine, valine, leucine, isoleucine, methionine, lysine, arginine, histidine, aspartic acid, asparagine, glutamic acid, glutamine, phenylalanine, tyrosine, tryptophan, proline), the achiral amino acid glycine, and the D-isomers of the chiral amino acids. “Amino acids” also include modified derivatives or analogs of the genetically encoded amino acids or their stereoisomers. Examples, of modified amino acids would be known to a person skilled in the art and include amino acids modified by phosphorylation, glycosylation, acylation, methylation, prenylation and the like.

“Polypeptide” includes amino acid polymers, including dipeptides, generally of less than about 50 000 amino acids. Unless the context dictates otherwise, the terms “polypeptide”, “peptide”, “oligopeptide”, and “protein” are used interchangeably herein. “Polypeptide” may refer to a single peptide or a collection of peptides, some of which may be covalently linked to other polypeptides, such as, for example through an intermolecular disulfide bond between cysteine residues on different peptides

Polypeptides may be chemically synthesized by known methods or be isolated and purified from natural or heterologous sources by known techniques. As would be appreciated by a person skilled in the art, polypeptides of fewer than about 50 amino acids are preferably prepared by conventional chemical synthesis. Polypeptides of more than about 100 amino acids may be preferably obtained by isolating the polypeptide from a natural or more preferably from a heterologous source, such as, for example, transgenic yeast or bacteria. Examples of heterologous sources include, but are not limited to, S. cerevisiae, P.pastoris and E. Coli.

In other embodiments, the bioactive agent may be a small molecule drug, including but not limited to: antibiotics, anti-viral agents, anesthetics, steroidal agents, anti-inflammatory agents, anti-neoplastic agents, antigens, vaccines, antibodies, decongestants, antihypertensives, sedatives, birth control agents, progestational agents, anti-cholinergics, analgesics, anti-depressants, anti-psychotics, diuretics, cardiovascular active agents, vasoactive agents, non-steroidal anti-inflammatory agents, nutritional agent and the like.

Methods for preparing PAI-graft-biodegradable polymer/bioactive agent polyplexes

In various embodiments, compositions comprising PAI-graft-biodegradable polymer/bioactive agent complexes may be prepared by mixing a solution of PAI-graft-biodegradable polymer with a solution of the bioactive agent. In a specific embodiment, the complex comprises PAI-graft-chitosan and a bioactive agent. A skilled person would also understand that the complex may be prepared by other methods, for example, by dissolving the bioactive agent in a PAI-graft-biodegradable polymer solution. When the bioactive agent carries a negative charge, it may be desirable to protonate the nitrogen atoms in the PAI-graft-polymer prior to contacting the PAI-graft-polymer with the bioactive agent, thereby providing a positively charged PAI-graft-polymer that can associate with negative charges present in the bioactive agent to form a complex by electrostatic attraction. As well, the monomers used to form the repeating unit may be selected to provide a PAI-graft-polymer with functional groups that are available to form covalent bonds with a bioactive agent. The PAI-graft-polymer may also form a complex by physically encapsulating the bioactive agent.

Preferably, PAI-graft-chitosan/bioactive agent polyplexes have physical dimensions that are compatible with endocytosis-mediated transfection. The polyplex may have an average size of about 200 nm, preferably about 150 nm to about 200 nm most preferably about 30 to about 150 nm. Preferably, PAI-graft-chitosan/DNA polyplexes have relatively uniform dimensions.

As would be appreciated by a person skilled in the art, the size of the polyplex may be influenced by a number of factors, including, but not limited to, the ionic strength of the solution comprising the polyplex. The average molecular size of a polyplex may be readily determined by methods known in the art, such as, for example, two photon fluorescence correlation spectroscopy (Clamme et al. (2003), Biophys J 84:196) or by dynamic light scattering (Erbacher et al. (1998), Pharmaceutical Research 15(9): 1332).

PAI-graft-biodegradable polymer/polynucleotide polyplexes, may be prepared by methods known to a person skilled in the art, for example, Boussif et al. ((1995), Proc. Nat. Acad. Sci. 92:7297). For polyplexes having PEI of at least about 22 kDa, the molar ratio of PEI nitrogen to DNA phosphate (N/P) in the PAI-graft-chitosan/DNA polyplex is preferably 6 to 30, more preferably 6 to 20 and most preferably 6 to 15 (Boussif et al. (1995), Proc. Nat. Acad. Sci. 92:7297). The N/P ratio is calculated on the basis of the poly(alkyleneimine) nitrogens per DNA phosphate and does not include the amino groups on the chitosan backbone. For PAI-graft-biodegradable polymer/DNA polyplexes with smaller grafted PAI chains, the N/P ratios are preferably greater. For example, for 1.2 kDa PEI, the N/P ratio may be as high as 50 (Petersen et al. (2002), Bioconjugate Chem. 13:812). Methods for determining appropriate N/P ratios would be known to a person skilled in the art. For instance, the optimal N/P ratio may be determined by gel retardation assays. Preferably, the N/P ratio is at least as great as the ratio required to show complete polynucleotide retardation in a gel retardation assay and at which the polyplex has a neutral or more preferably a positive zeta potential. Without being restricted to any particular theory, it is believed that positively charged polyplexes may enhance transfection efficiency by electrostatically interacting the with negatively charged phospholipid head groups on the surface of a target cell.

Preferably, the polynucleotide used to prepare a PAI-graft-biodegradable polymer/polynucleotide polyplex is substantially pure. A polynucleotide preparation is “substantially pure” if it comprises at least 50%, preferably at least 80%, more preferably at least 90% and most preferably at least 95% of the polymers in the preparation. Polynucleotides may be provided by standard techniques known to those skilled in the art and described, for example in Sambrook et al. in Molecular Cloning: A Laboratory Manual, 3^{rd} Edition, Cold Spring Harbour, Laboratory Press and other laboratory manuals. In various asects, the polynucleotides may be chemically synthesized using techniques such as are disclosed, for example, in Itakura et al. U.S. Pat. No. 4,598,049; Caruthers et a.l U.S. Pat. No. 4,458,066; and Itakura et al. U.S. Pat. Nos. 4,401,796 and 4,373,071. Alternatively, or in addition, the polynucleotide may be obtained from natural sources and purified from contaminating components found normally in nature.

In various embodiments, polyplexes are formed by adding a PAI-graft-chitosan solution to a polynucleotide solution. The admixed solutions may be incubated under conditions sufficient to form a stable PAI-graft-chitosan/polynucleotide polyplex, for, such as, for example, for 10 to 30 minutes at room temperature. The incubation period may optionally be interrupted by one or more vortexing steps. Without being limited to any particularly theory, the polyplex is believed to form as a consequence of the electrostatic interactions between the positively charged PAI amino groups in the PAI-graft-chitosan polymer and the negatively charged phosphate backbone of the polynucleotide.

The formation of stable PAI-graft-biodegradable polymer/polynucleotide polyplexes may be readily determined by methods known in the art, such as, for example, by the ability of the PAI-graft-chitosan to inhibit or diminish the electrophoretic mobility of the polynucleotide in an agarose gel.

Other PAI-graft-biodegradable polymer/bioactive agent polyplexes, such as, for example, PAI-graft-biodegradable polymer/RNA polyplexes, PAI-graft-biodegradable polymer/polypeptide polyplexes and PAI-graft-biodegradable polymer/drug polyplexes may be prepared by analogous methods.

Methods of Delivering Bioactive Agents

The PAI-graft-biodegradable polymer/bioactive agent polyplexes according to different embodiments may be used to deliver the bioactive agent into a target cell. Preferably, the bioactive agent is a polynucleotide, more preferably DNA, even more preferably a DNA expression vector. In specific embodiments, the expression vector encodes a therapeutic or a marker molecule, preferably a therapeutic protein or a marker protein.

In different embodiments, the bioactive agent is delivered to a target cell by contacting the PAI-graft-biodegradable polymer/bioactive agent polyplex with the target cell. In specific embodiments, the target cell is a eukaryotic cell, preferably a vertebrate cell, more preferably a mammalian cell and most preferably a human cell. In various embodiments, the PAI-graft-biodegradable polymer/DNA polyplex is contacted with the target cell by providing the PAI-graft-biodegradable polymer/DNA polyplexes to a medium containing the target cells. In specific embodiments, the PAI-graft-biodegradable polymer is PAI-graft-chitosan.

The transfection efficiency of the PAI-graft-biodegradable polymer/DNA polyplexes may be determined by methods known in the art. Preferably, the DNA in the polyplex encodes a polypeptide whose expression can be readily determined. More preferably, the expression level of the polypeptide is readily quantitatively determined. For example, the DNA may encode a marker protein whose presence or catalytic activity may be determined by optical methods such as, for example, green fluorescent protein, β-galactosidase or luciferase. Other marker proteins would be known to a person skilled in the art. In a specific embodiment, the polyplex comprises an expression vector encoding a luciferase marker protein.

In various embodiments, the PAI-graft-biodegradable polymer/DNA polyplex may further comprise a targeting element. The targeting element may bind to receptors on the surface of target cells and increase the transfection efficiency of the polyplex in cells expressing the appropriate receptor (Schneider et al. (1996), Nucl Acid Res. 24:3873).

It will be appreciated that other than the N/P ratio, other parameters, such as ionic strength of the polyplex solution, the DNA concentration, the protocol of complex formation, the average molecular weight of the poly(alkyleneimine), the zeta potential and polydispersity of the polyplex may strongly influence transfection efficiency. (Kichler (2004) J. Gene Med. 6:S3-S10). A person skilled in the art would understand how to vary these parameters in order to increase transfection efficiency.

Compositions containing a PAI-graft-biodegradable polymer/polynucleotide polyplex of the invention may optionally contain other transfection-facilitating compounds. A number of such compositions are described in WO 93/18759, WO 93/19768, WO 94/25608, and WO 95/02397. They include spermine derivatives useful for facilitating the transport of DNA through the nuclear membrane (see, for example, WO 93/18759) membrane-permeabilizing compounds such as GALA, Gramicidine S, and cationic bile salts (see, for example, WO 93/19768), and non-ionic surfactants such as Pluronic-block copolymers (Kuo et al. (2003), Biotech Appl. Biochem 37:267). In these embodiments, the transfection-facilitating compounds are preferably added to a solution containing the polynucleotide prior to adding the PAI-graft-biodegradable polymer (Kuo et al. (2003) Biotech Appl. Biochem 37:267).

Pharmaceutical Preparations

The invention also provides pharmaceutical compositions comprising PAI-graft-PAI-graft-biodegradable polymer bioactive agent polyplex and a pharmacologically acceptable excipient or carrier. The pharmaceutical composition may be soluble in an aqueous solution at a physiologically acceptable pH.

As used herein “pharmaceutically acceptable carrier” or “excipient” includes any and all solvents, dispersion media, coatings, antibacterial and antifungal agents, isotonic and absorption delaying agents, and the like that are physiologically compatible. In one embodiment, the carrier is suitable for parenteral administration. Alternatively, the carrier can be suitable for intravenous, intraperitoneal, intramuscular, sublingual or oral administration. The proportion and identity of the pharmaceutically acceptable carriers or excipients is determined by chosen route of administration, compatibility with the vector and standard pharmaceutical practice. Generally, the pharmaceutical composition will be formulated with components that will not significantly impair the biological activities of the vector. Suitable vehicles and diluents are described, for example, in Remington's Pharmaceutical Sciences (Remington's Pharmaceutical Sciences, Mack Publishing Company, Easton, Pa., USA 1985). Pharmaceutically acceptable carriers include sterile aqueous solutions or dispersions and sterile powders for the extemporaneous preparation of sterile injectable solutions or dispersions. The use of such pharmaceutically acceptable carriers and excipients for pharmaceutically active substances is well known in the art. Except insofar as any conventional pharmaceutically acceptable carriers and excipients is incompatible with the active compound, use thereof in the pharmaceutical compositions of the invention is contemplated. Supplementary active compounds can also be incorporated into the compositions.

Pharmaceutical compositions typically must be sterile and stable under the conditions of manufacture and storage. Conventional procedures and ingredients for the selection and preparation of suitable formulations are described, for example, in Remington's Pharmaceutical Sciences and in The United States Pharmacopeia: The National Formulary (USP 24 NF 19) published in 1999. The composition can be formulated as a solution, microemulsion, liposome, freeze-dried powder, spray-dried powder or other ordered structure suitable to high drug concentration. In specific embodiments the composition is spray-dried or freeze-dried. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. In many cases, it will be preferable to include isotonic agents, for example, sugars, polyalcohols such as mannitol, sorbitol, or sodium chloride in the composition. Prolonged absorption of the injectable compositions can be brought about by including in the composition an agent which delays absorption, for example, monostearate salts and gelatin. Moreover, a poly(amino ester)-agent complex can be administered in a time release formulation, for example in a composition which includes a slow release polymer. The active compounds can be prepared with carriers that will protect the compound against rapid release, such as a controlled release formulation, including implants and microencapsulated delivery systems. For this purpose, biodegradable, biocompatible polymers can be used, including but not limited to ethylene vinyl acetate, polyanhydrides, polyglycolic acid, collagen, polyorthoesters, polylactic acid and polylactic, polyglycolic copolymers (PLG). Many methods for the preparation of such formulations are patented or generally known to those skilled in the art.

Sterile injectable solutions can be prepared by incorporating the PAI-graft-biodegradable polymer/bioactive agent polyplex in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying, freeze-drying and spray-drying which yields a powder of the active ingredient plus any additional desired ingredient from a previously sterile-filtered solution thereof. In accordance with an alternative aspect of the invention, a PAI-graft-biodegradable polymer/bioactive agent polyplex may be formulated with one or more additional compounds that enhance the solubility of the PAI-graft-polymer/bioactive agent complex.

In various embodiments, the bioactive agent is protein or a drug or preferably is an expression vector encoding a therapeutic product. “Therapeutic product” as used herein describes any product that effects a desired therapeutic result, for example treatment, prevention or amelioration of a disease. “Therapeutic product” includes prophylactic products that effect a desired prophylactic result, such as preventing or inhibiting the rate of various disease onsets or progressions. In different embodiments, the therapeutic product may be a therapeutic protein, a therapeutic peptide or a therapeutic RNA, for example, a small interfering RNA (siRNA) or an anti-sense RNA capable of hybridizing to a specific target nucleic acid sequence with the cell.

Depending on the intended mode of administration, the pharmaceutical compositions may be in the form of solid, semi-solid or liquid dosage forms, such as, for example, tablets, suppositories, pills, capsules, powders, liquids, suspensions, lotions, creams, gels, or the like, preferably in unit dosage forms suitable for single administration of a precise dosage. The composition can include, as noted above, an effective amount of the selected bioactive agent in combination with a pharmaceutically acceptable carrier and, in addition, may include other medicinal agents, pharmaceutical agents, carriers, adjuvants, diluents, and the like.

In various embodiments, the invention provides corresponding methods of medical treatment, in which a therapeutically effective amount of a PAI-graft-chitosan/bioactive agent polyplex is administered in a pharmacologically acceptable formulation to a patient or subject in need thereof.

In various embodiments, a PAI-graft-biodegradable polymer/bioactive agent polyplex is administered to a patient or subject in an effective amount and dosage and for a sufficient time to achieve a desired result. For example, the vectors may be administered in quantities and dosages necessary to deliver a therapeutic gene encoding a product which functions to alleviate, improve, mitigate, ameliorate, stabilize, prevent the spread of, slow or delay the progression of or cure a disease or disorder. In specific embodiments, the bioactive agent is an expression vector encoding a therapeutic product.

The effective amount to be administered to a patient can vary depending on many factors such as, among other things, the pharmacodynamic properties of the vector, the mode of administration, the age, health and weight of the subject, the nature and extent of the disorder or disease state, the frequency of the treatment and the type of concurrent treatment, if any.

The administration in vivo can be performed by parenteral administration, such as, for example, by intravenous injection including regional perfusion through a blood vessel supplying the tissue(s) or organ(s) having the target cell(s). Other means of administration can include inhalation of an aerosol, subcutaneous, intraperitoneal, or intramuscular injection, direct transfection into cells prepared for transplantation into an organ that is subsequently transplanted into the subject. Further administration methods can include oral administration, particularly when the complex is encapsulated, or rectal administration, particularly when the complex is in suppository form.

Various embodiments of the invention may have wide application in gene therapy of various diseases such as, for example, cancer, neurological disorders, cardiovascular disorders, and AIDS. In other embodiments, the invention may have application in the in vitro or in vivo delivery of non-therapeutic agents, such as, for example, diagnostic agents.

All documents referred to herein are fully incorporated by reference.

Although various embodiments of the invention are disclosed herein, many adaptations and modifications may be made within the scope of the invention in accordance with the common general knowledge of those skilled in the art. Such modifications include the substitution of known equivalents for any aspect of the invention in order to achieve the same result in substantially the same way. All technical and scientific terms used herein have the same meaning as commonly understood by one of ordinary skill in the art of this invention, unless defined otherwise.

The word “comprising” is used as an open-ended term, substantially equivalent to the phrase “including, but not limited to”. Singular articles such as “a” and “the” in the specification incorporate, unless the context dictates otherwise, both the singular and the plural.

The following examples are illustrative of various aspects of the invention, and do not limit the broad aspects of the invention as disclosed herein.

### EXAMPLES

Materials and Reagents

Plasmid DNA (pCMV-Luc) was obtained from Elim Biopharmaceuticals, Inc, CA, USA). Plasmid VR1255 is a plasmid encoding the firefly luciferase with a size of 6.4 kb driven by the cytomegalovirus (CMV) promoter/enhancer.

Chitosan (low molecular weight, 75-85% deacetylation, viscosity (Brookfield, 1% acetic acid) 20-200 cps.) and MTT (3-(4,5-dimethyl-thiazol-2-yl)-2,5-diphenyl tetrazolium bromide) were purchased from Aldrich (Milwaukee, Wis., USA) and used without further purification.

Ethanolamine, hydrochloric acid, sulphuric acid, and other materials, including solvents, were used as received, i.e. without further purification.

General Characterization

¹H NMR (400 MHz) and ¹³C NMR (100 MHz) were recorded on a Bruker DRX-400 spectrometer. Gel permeation chromatography (GPC) was carried out on a Waters 2690 apparatus with a column (Waters Ultrahydrogel 500 and 250) and a Waters 410 refractive index detector using 0.5 M acetic acid/0.5 M sodium acetate as the eluent at a flow rate of 1.0 ml/min. The molecular weights were calibrated against poly(ethylene oxide) standards.

Preparation of Water Soluble Chitosan

25 g chitosan powder was dispersed in 500 ml 0.5% H₂O₂ in an autoclave vessel. After purging with argon for 5 min, the vessel was sealed. Then the reaction was performed under around 90 C and 100 psi for 1 hour. The filtered solution was precipitated in acetone. The powder collected was dissolved in water and the solution was lyophilized to chitosan powder.

### Example 1

### Synthesis and Characterization of PEI-Graft-Chitosan

0.4 g of pure chitosan powder was dissolved in 5 ml of de-ionized water, and hydrochloric acid (HCl) with the molar ratio of HCl to amine in chitosan to be 1:10 was added. After ethyleneimine (EI) with molar ratio of EI to amine in chitosan to be 5:1 was added dropwise into the solution under stirring, the polymerization was performed at ambient temperature for 5 days. Finally, the temperature was increased to 60° C. for one day. After being purified by dialysis in water, the solution was lyophilized to give a light yellow powder.

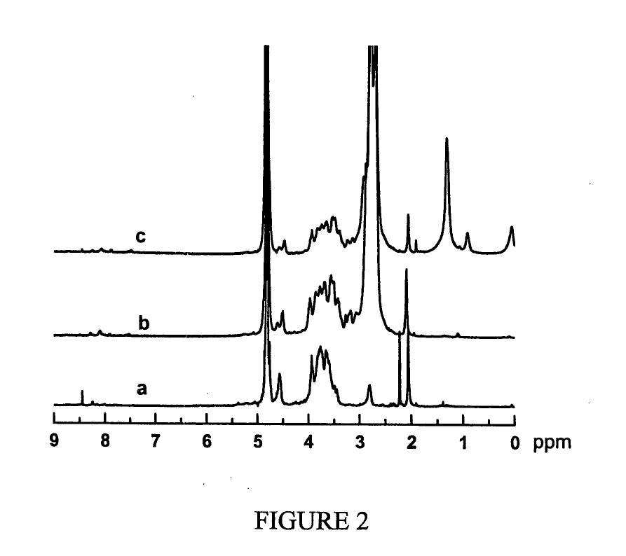

The chemical structure of PEI-graft-chitosan was analysed by ¹H and ¹³C NMR. FIGS. 1a and 2a show the ¹H and ¹³C NMR spectrum of PEI-graft-chitosan respectively. From ¹H-NMR spectrum (as shown in FIG. 1a), the ratio of PEI/the repeating units in chitosan is calculated to be around 4.8:1 close to the feed molar ratio, based on the integral intensities of peak located at 2.5-3.0 and 3.2-4.2 ppm related to the proton in PEI and attached to carbons 2 to 6 in chitosan, respectively.

In the 13C-NMR spectrum of PEI-graft-chitosan (as shown in FIG. 2b), the peaks has been assigned to different types of carbons adjacent to different types of amines based (Harpe, A V; Petersen, H.; Li, Y X.; Kiseel, T. J. Controlled Release 69, 309 (2000)). Based on inverse-gated broadband decoupled ¹³C-NMR peak areas, the molar ratio of different type of primary: secondary: tertiary amines is approximately 1:1.97:1.23.

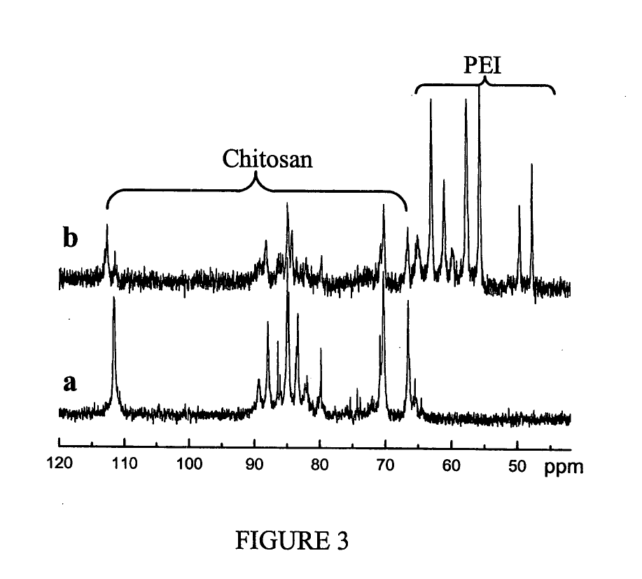

The molecular weight of PEI-graft-chitosan was characterized by GPC and the results are shown in FIG. 3. After being grafted with PEI, the number average molecular weight (Mn) increases from 2516 to 3190 with the polydispersity index of molecular weight increasing from 1.29 to 1.49.

### Example 2

### Modification of PEI-Graft-Chitosan by Attaching Hexadecane.

0.1 g PEI-graft-chitosan was dissolved in 4 ml chloroform. After exchanging with nitrogen by freezing and thawing for three times, 0.037 ml 1-idohexadecane was added after 0.02 ml triethylamine was introduced into the solution under stirring. The reaction was performed at 55° C. for 6 h followed by being kept at ambient temperature for 24 h. After removing chloroform, a powder was obtained. Then the powder was dissolved in 10% ethanol/water and purified by dialysis in 10% ethanol/water. A fine powder was obtained by lyophilization.

FIG. 1c is ¹H-NMR spectrum of the hexadecane-graft-PEI-graft-PEI. The molar ratio of hexadecane/PEI is approximately 1:7 based on the integral intensities of peaks of protons of hexadecane and PEI at 0.7-1.5 ppm and 2.5-3.0 ppm, respectively.

GPC profile of hexadecane-graft-PEI-graft-chitosan is shown in FIG. 3c. Due to the attachment of hydrophobic long alkyl chains, hexadecane-graft-PEI-graft-chitosan aggregates in aqueous solution. Mn measured by GPC relative to PEO standards decreased to 3190.

### Example 3

### Formation and Analysis of DNA/PEI-Graft-Chitosan and DNA/Hexadecane—graft-PEI-Graft-Chitosan Complexes

Plasmid DNA (pCMV-Luc) was diluted to the chosen concentration (usually 0.5-2.0 μg/μl) in pure water under vortexing. Various amounts of 0.1 M solution of PEI-graft-polymer or hexadecane-PEI-graft-chitosan in water were added slowly to the DNA solutions. The amount of polymer added was calculated based on chosen N/P ratios of PEI-graft-polymer:DNA. After the solution was incubated at ambient temperature for 30 min with gentle vortexing, the formed PEI-graft-polymer/DNA polyplexes was mixed with a loading buffer and loaded onto a 1% agarose gel containing ethidium bromide. Gel electrophoresis was run at room temperature in TBE buffer at 80 V for 60 min. DNA bands were visualized by an UV (254 nm) illuminator.

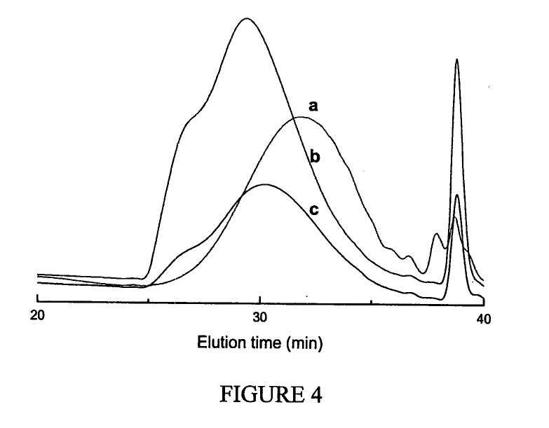

The results of the agarose gel electrophoresis, shown in FIG. 4a, demonstrate that the migration of DNA was retarded completely when the N/P ratios of PEI-graft-chitosan/DNA was around 2.5/1. But the oligo-chitosan cannot form stable complexes with DNA even at N/P ratios equal or greater than 140. This phenomena is reasonable because it has been verified that only chitosan with a molecular weight higher than about 4056 has enough condensation capability to form stable complexes with DNA (Köping-Höggård et al (2003) J Gene Med. 5: 130).

The condensation capability of PEI is also related to its molecular weight. For example, for PEI of a molecular weight of around 5000, two times higher N/P ratio is needed to form stable complexes with DNA as compared with 25 k PEI (Kunath, K.; Harper, A. V.; Fisher, D.; Petersen, H.; Bickel, U.; Voigt, K.; Kisse; T. J. of Controlled Release, 89, 113-125 (2003). Herein the Mₙ of PEI-graft-chitosan was determined to be around 4000, but stable complexes with DNA could be formed with the N/P ratio to be around 2.5:1 showing its good condensation capability close to 25k PEI. FIG. 4b reflects that attaching hexadecane to PEI-graft-chitosan reduces the condensation capability.

### Example 4

### Cytotoxicity of PEI-Graft-Chitosan

HeLa cells (8000 cells/well) were seeded in a 96-well plate. The cells were incubated for 4 h with 200 ul of complete DMEM containing polymers, or PLL or PEI at different concentrations. After 4 h, the medium in each well was replaced with 100 ul of fresh complete medium. Then 25 ul of MTT solution in PBS (5 mg/ml) was added to reach the final concentration of 1 mg/ml and cells were incubated for another 2 hours. After 2 h, 100 ul of the extraction buffer (20% SDS in 50% DMF, pH 4.7) was added to the wells and cells were incubated overnight. The optical densities were measured at 550 nm using a microplate reader (Model 550, Bio-Rad Lab. Hercules, Calif.). Values were expressed as a percentage of the control to which no polymers had been added

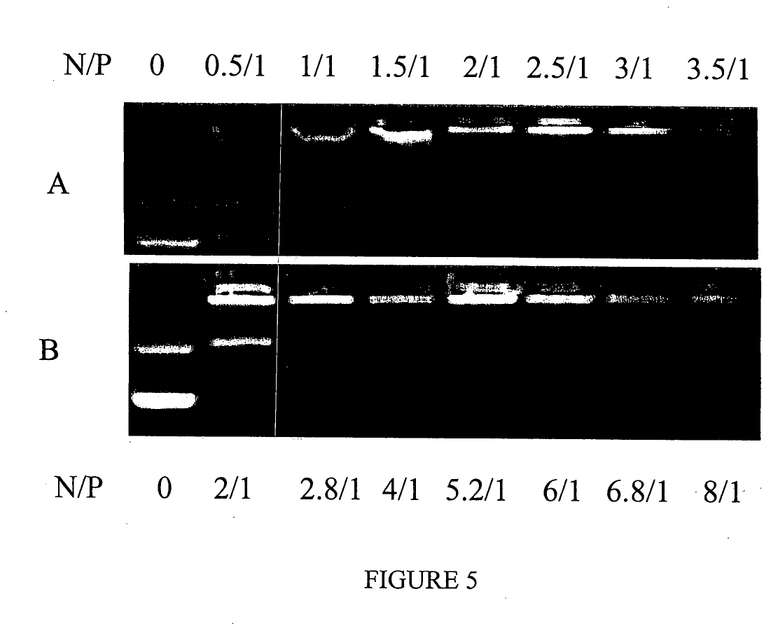

FIG. 5 shows the results of the cytotoxicity assay. The cytotoxicity of PEI-graft-chitosan is much lower than that of 25 k PEI, its LD₅₀ (97.3 μg/ml) being around 6 times higher that that of 25 K PEI (13.5 μg/ml). Also it reflects that attaching hexadecane has no significant affects on the cytotoxicity profile of PEI-graft-chitosan with a LD50 to be 110.9 ug/ml.

### Example 5

### Cell Transfection Efficiency

The in vitro transfection efficiency of PEI-graft-chitosan was evaluated in HeLa cells using the complexes formed between PEI-graft-chitosan and pVR 1255 DNA. Cells were seeded 24 h prior to transfection into 24-well plates (Becton-Dickinson, Lincoln Park, N.J) at a density of 8×10⁴ per well with 1 ml of indicated medium. At the time of transfection, the medium in each well was replaced with 300 μl of Opti-MEM. The complexes of polymer/DNA were incubated with the cells for 4 h at 37° C. The medium was replaced with 1 ml of fresh complete medium and cells were further incubated for 44 h. After the incubation, cells were permeabilized with 200 μl of cell lysis buffer (Promega Co., Wis.). After two cycles of freezing and thawing, the lysate was transferred into mico reaction tubes and centrifuged for 5 min. Then the luciferase activity of the supernatants were measured using 100 ul luciferase assay reagent (Promega Co., Madison, Wis.) on a single-well luminometer (Berthold Lumat LB 9507, Germany) for 10 s. The light units (LU), measured using a protein assay kit (Bio-Rad Labs, Hercules, Calif.), were normalized against protein concentration in the cell extracts. Luciferase activity was expressed as relative light units (RLU ng/mg protein). The data were reported for duplicate samples.

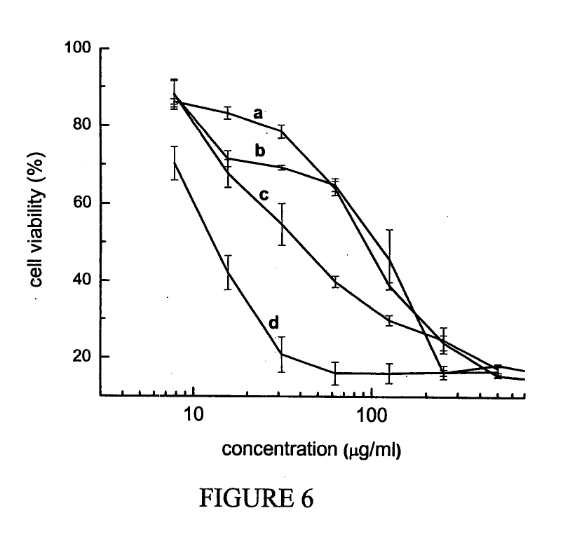

FIG. 6 displays the results for the complexes comprised of different N/P ratios. For PEI-graft-chitosan, higher N/P ratios lead to higher transfection efficiency up to the N/P ratio of 40. For PEI, the optimal N/P ratio is about 10, and higher N/P ratios result in poor transfection efficiency, possibly due to the higher cytotoxicity of PEI relative to PEI-graft-chitosan. Remarkably, PEI-graft-chitosan has a transfection efficiency close to 25 k PEI when the N/P ratio is around 10, and has a five time higher transfection efficiency than 25 k PEI when the N/P ratio is increased to 40.

When considered in the context of its low cytotoxicity and biodegradability, PEI-graft-chitosan is a promising material for the preparation of safe and efficient vectors for the delivery of DNA. The hexadecane-PEI-graft-chitosan also has good transfection efficiency, but including the hexadecane had no improvement in the transfection efficiency of the polyplex.

Wistar rats (male, 200 to 250 g, 20˜24 per group) were used for the in vivo transfection efficiency evaluation. Six to eight-week-old male Wistar rats were obtained and housed in National University of Singapore Animal Holding Unit. Rats were maintained on ad libitum rodent feed and water at room temperature, 40% humidity. All animal procedures were approved by the National University of Singapore Faculty of Medicine Animal Care and Use Committee. The complexes of PEI-chitosan and VR1255 plasmid encoding firefly luciferase driven by CMV promoter were prepared with N/r ratio (molar ratio of amino group in chitosan to phosphate group in DNA) being 5:1; 10:1; 20:1; 40:1, respectively, for evaluation. The complexes of chitosan and DNA (N/P=3:1), PEI (25 K) and DNA (N/P ratio=10:1), and naked DNA were used as control experiments. Animals were laparotomized under general anesthesia and the liver was then surgically isolated from the surrounding tissue. The complexes of PEI-graft-chitosan or naked DNA were administered at the dose equivalent to 200 μg of plasmid (˜0.8 mg/kg of body weight) in 4 ml of medium into the common bile duct over 20 minutes (0.2 ml/min) using a syringe pump and a 33 Gauge needle. The 33G needle was inserted into the common bile duct and a tie was used to secure the needle. A tie was then placed around the bile duct between the liver and the point of infusion to prevent back flow, and the needle was withdrawn. After 30 min, all ties were removed. The needle hole in the bile duct might require a single 10-O nylon (Ethicon) stitch to prevent bile leakage, if necessary. Rats were kept on normal diet. After 3 days, five rats from each group were sacrificed, and major organs (liver, heart, lung, spleen and kidney) were harvested and stored at −80° C. Each liver was divided into 4 sections composed of median, left, right, and caudate lobes. 2 ml of lysis buffer (0.1% Triton X-100, 2 mmol/L ethylenediaminetetraacetic acid, and 0.1 mol/L Tris-HCl pH 7.8) per organ gram weight was used on each sample, and major organs were homogenized and then subjected to two cycles of freeze-thawing, and centrifuged at 14,000 rpm for 10 minutes. The samples protein concentration in the supernatant was determined by using a luciferase protein assay kit (Pierce, Rockford, Ill.).

A total of 10 μL of supernatant was analyzed for luciferase activity. Luminescence was measured for 10 seconds of incubation, and the luciferase activity for each assay was presented as relative light units per gram of tissue. The mean of luciferase activity of the liver was the sum of the values obtained by timing the relative light units per gram of each lobe with the weight percentage of each lobe relative to the total liver weight.

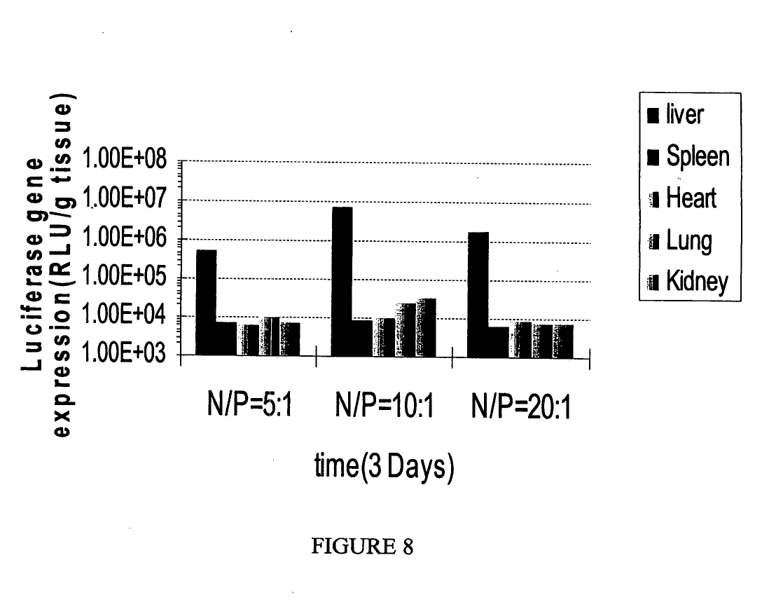

FIG. 8 shows that the gene expression in kidney, lung, heart, and spleen were negligible in comparison with that in liver.

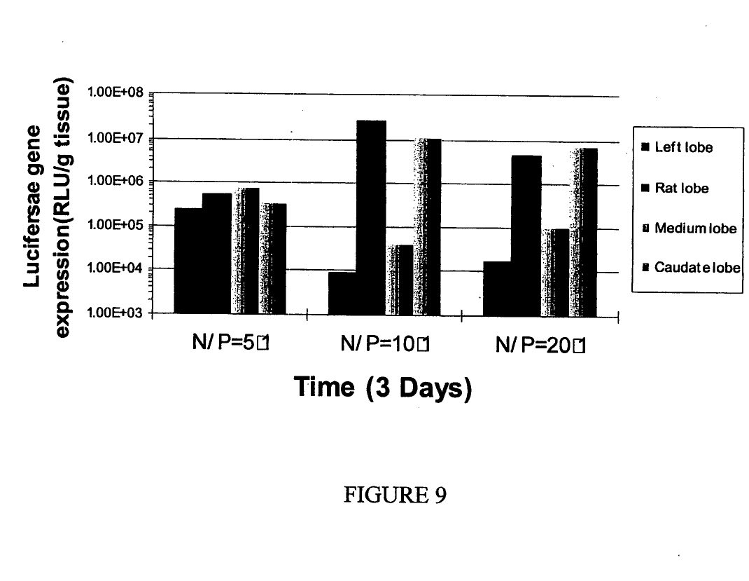

FIG. 9 shows the distribution of the transgene product in different lobes of the transfected rat livers following intrabiliary infusion. Luciferase expression in left lobe and medium lobe were about 5-1000 times lower than other lobes of the liver at the early time points for the complexes with N/P ratios being 10:1 or 20:1, however, the expression levels among different lobes for the complexes with a N/P ratio of 5:1 are probably due to the aggregation of the complexes in bile duct and canaliculi.

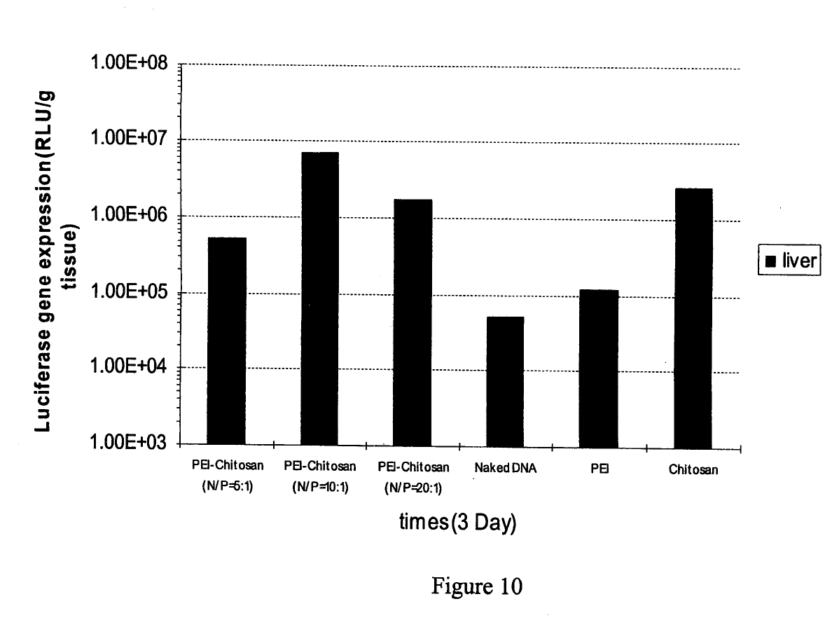

FIG. 10 shows that the complexes of PEI-chitosan/DNA (N/P=10:1) have about 141 times higher transfection efficiency than naked DNA, 58 times higher transfection efficiency than a PEI-complex (N/P=10:1), and 3 times higher transfection efficiency than a chitosan-complex (N/P=3:1) in a same time point.

### Example 6

### Preparation of N-CBz-poly(L-aspartic acid-co PEG)

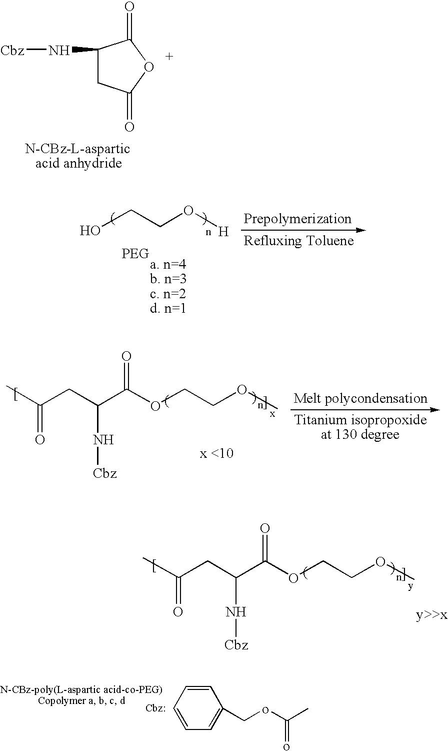

N-CBz-L-aspartic anhydride (2.0 g, 8 mmol), PEG (8 mmol), p-Toluenesulfonic acid monohydrate (1.8 mg, 9.6×10⁻³ mmol) and 50 mL of toluene were placed into a 100 mL round-bottom flask equipped with a magnetic stirrer, a Dean-Stark trap with a reflux condenser and Argon inlet. The refluxing was maintained for 48 h and then cooled to room temperature

After evaporation of solvent under vacuum a sticky liquid pre-polymer was obtained. To this sticky product was added 0.5 wt. % of titanium isopropoxide. The mixture was stirred under vacuum at 130° C. for 6 h and then cooled to room temperature.

The resulting product was dissolved in chloroform and those insoluble materials were filtered out. The organic solvent was combined and concentrated to 5 mL and precipitated into 10-fold excess of ether. The precipitate was dried at 50° C. under vacuum overnight to get the final product. Yields range between 80-85%.

### Example 7

### Preparation of poly(L-aspartic acid-co-PEG)

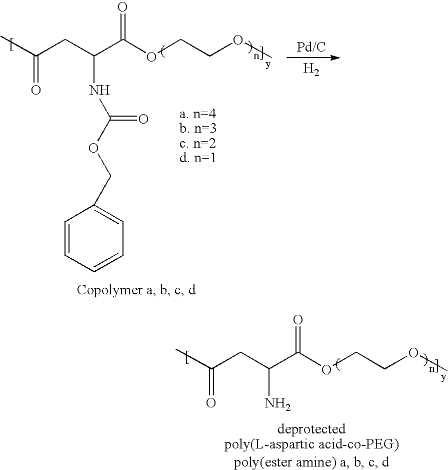

A 10% Pd/C catalyst (0.25 g) was used with H₂ in DMF (7.5 mL) solution of the L-aspartic acid-co-PEG polymer (0.25 g) and the solution was stirred for 24 h at room temperature and then filtered to remove the catalyst which was continued to be washed for 2 more times by DMF (7.5 mL×2). The combined solution was concentrated to ¼ volume under vacuum at room temperature and then precipitated into 10-fold excess of acetone. The precipitate was dried under vacuum at 50° C. to get the final product.

Yields range between 66-72%.

### Example 8

### Preparation of PEI-graft-poly(L-aspartic acid-co-PEG)

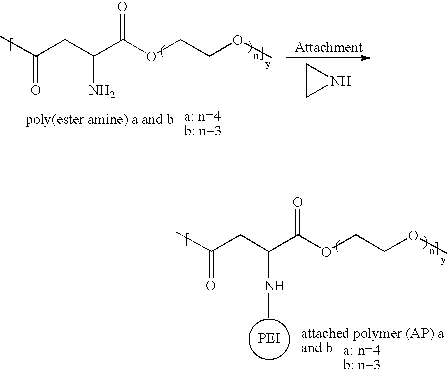

To a solution of the deprotected poly(L-aspartic acid-co-PEG) in DMF (8.0×10⁻¹ mmol dissolved in 8 mL of DMF) was added 160 μl (4.0×10⁻² mmol) of HCl/diethylether (0.25M) and stirred for 5 minutes and then ethyleneimine (210.0 μL, 4.0 mmol) was added. The above solution was stirred for 48 h at room temperature and then the solution was concentrated to one quarter volume under vacuum and precipitated into 10-fold excess of acetone. The precipitate was dried at 50° C. under vacuum to get the final product, in which at least 10% of the aspratic acid amino groups are grafted with PEI. Yields range between 60-64%.

1. A poly(alkyleneimine)-graft-biodegradable polymer comprising at least two poly(C₂-C₆ alkyleneimine) side chains and a biodegradable polymer main chain, wherein each of said at least two side chains is linked to said main chain by a single covalent bond.

2. The poly(alkyleneimine)-graft-biodegradable polymer according to claim 1, wherein the biodegradable polymer is a polysaccharide, polyester, polyamide, poly(ester amide), poly(ester carbonate), poly(ester ether), poly(ester urethane), polypeptide, polyurethane, polyether, polyphosphoester, poly(phosphazenes) homopolymer or a copolymer of one or more thereof.

3. The poly(alkyleneimine)-graft-biodegradable polymer according to claim 2 wherein the biodegradable polymer is poly(L-aspartic acid-co-PEG).

4. The poly(alkyleneimine)-graft-biodegradable polymer according to claim 3 wherein the biodegradable polymer has about 2 to about 10000 repeating units.

5. The poly(alkyleneimine)-graft-biodegradable polymer according to claim 4 wherein the poly(C₂-C₆ alkyleneimine) side chain is a poly(ethyleneimine) side chain.

6. The poly(alkyleneimine)-graft-biodegradable polymer according to claim 5 wherein the poly(ethyleneimine) side chain comprises about 2 to about 2325 ethyleneimine repeating units.

7. The poly(alkyleneimine)-graft-biodegradable polymer according to claim 6 further comprising a targeting element.

8. The poly(alkyleneimine)-graft-biodegradable polymer according to claim 7 wherein the targeting agent is a polypeptide, carbohydrate, glycopolypeptide, lipopolypeptide, low-density lipoprotein (LDL), lipid, steroid, C₅₋₂₀ alkyl group, antibody or fragment thereof.

9. A poly(alkyleneimine)-graft-biodegradable polymer comprising the structure of formula I: 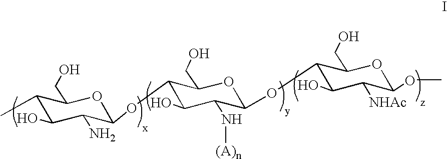
wherein:
x+y+z is 2 to about 1000;
x/(x+y+z) is 0 to about 99.9%;
y is at least 2;
z/(x+y+z) is 0 to about 60%; and
(A)ₙ is a poly(C₂-C₆ alkyleneimine) having n repeating units.

10. The polymer according to claim 9 wherein:
(x+y+z) is about 3 to about 20; and
y/(x+y+z) is about 10% to about 90%.

11. The polymer according to claim 10 wherein (A)ₙ is poly(ethyleneimine).

12. The polymer according to claim 11 wherein n is about 2 to about 2325.

13. The polymer according to claim 12 further comprising a targeting element.

14. A method of preparing a PAI-graft-biodegradable polymer comprising the step of reacting a C₂-C₆ alkyleneimine and a biodegradable polymer under acidic conditions.

15. The method according to claim 14 wherein the C₂-C₆ alkyleneimine is ethyleneimine.

16. The method according to claim 15 wherein the biodegradable polymer is chitosan or poly(L-aspartic acid-co-PEG).

17. The method according to claim 16 wherein the chitosan has a degree of deacetylation of about 75% to about 85%.

18. The method according to claim 17 wherein a solution comprising ethyleneimine is added to an acidified solution comprising chitosan.

19. The method according to claim 18 wherein the solution comprising ethyleneimine and the acidified solution comprising chitosan are added in a molar ratio of ethyleneimine to chitosan amine of about 5:1.

20. A composition comprising the poly(alkyleneimine)-graft-biodegradable polymer according to claim 1 and a bioactive agent.

21. A composition comprising the poly(alkyleneimine)-graft-biodegradable polymer according to claim 11 and a bioactive agent.

22. The composition according to claim 21 wherein the bioactive agent is DNA.

23. The composition according to claim 22 wherein the DNA encodes a therapeutic molecule.

24. The composition according to claim 23 wherein DNA encodes a marker molecule.

25. The composition according to claim 23 further comprising a pharmaceutically acceptable carrier.
